(19) United States
(12) Patent Application Publication
(10) Pub. No.: US 2013/0289406 A1
(43) Pub. Date: Oct. 31, 2013

(54) Ultrasonographic Systems For Examining And Treating Spinal Conditions

(71) Applicant: Christopher Schlenger, Stockton, CA (US)

(72) Inventor: Christopher Schlenger, Stockton, CA (US)

(21) Appl. No.: 13/713,256

(22) Filed: Dec. 13, 2012

Related U.S. Application Data
(60) Provisional application No. 61/640,561, filed on Apr. 30, 2012.

(51) Int. Cl. A61B 8/08; A61B 8/00; A61B 8/14

(52) U.S. Cl. CPC A61B 8/0875; A61B 8/14; A61B 8/5207; A61B 8/461; A61B 8/4263; A61B 8/466; A61B 8/483
USPC 600/443

(57) ABSTRACT

The present disclosure is directed to an ultrasonographic imaging system for examining and treating spinal conditions. In some examples, the system includes an ultrasound transducer probe configured to captured an ultrasound image, an ultrasound processor configured to receive data from the ultrasound transducer probe, a video capture card configured to receive the digital image from the ultrasound processor, an image processing system configured to process the digital image, the image processing system further including an image restoration module, an algorithm module, and a visualization module, a plurality of optical targets and a display device.

### CROSS REFERENCE TO RELATED APPLICATIONS

This application claims priority to copending U.S. Application Ser. No. 61/640,561, filed on Apr. 30, 2012, which is hereby incorporated by reference for all purposes.

### BACKGROUND

The present disclosure relates generally to ultrasound imaging systems. In particular, ultrasonographic systems for examining and treating spinal conditions are described.

Various techniques for acquiring images of subcutaneous body structures, such as tendons, muscles, vessels, internal organs, and bone surfaces, are in use today by medical practitioners. Known techniques include x-ray, magnetic resonance imaging (MRI), and radionuclide imaging. X-ray and radionuclide imaging techniques suffer from the drawback of exposing patients to potentially harmful ionizing radiation. MRI techniques can be expensive and therefore unavailable to some patients as a diagnostic tool. Further, MRI is unsatisfactorily slow and provides low resolution images of bone structures.

Another technique for imaging subcutaneous body structures as a diagnostic aid involves using ultrasonographic systems. Ultrasound devices used in ultrasonographic systems produce sound waves at a frequency above the audible range of human hearing, which is approximately 20 kHz. Sound waves between 2 and 18 Mhz are often used for ultrasound medical diagnostic applications. At present, there are no known long term side effects from interrogating the human body with ultrasound waves.

Acquiring images of the spine is one known application for ultrasonographic systems. However, known ultrasonographic systems for spinal examination and treatment are not entirely satisfactory for the range of applications in which they are employed.

For example, existing ultrasound technology does not have the ability to readily recreate three dimensional representations of bone structures. Further, conventional ultrasound systems do not enable the user to re-identify the position of bone structures externally. Moreover, current ultrasonographic systems generally only represent soft tissue structures in three dimensional space and do not satisfactorily represent bone structures in three dimensions.

Further limitations of conventional ultrasonographic systems relate to their reliance on magnetic positioning systems as opposed to more precise optical tracking systems to determine the patient's and/or the ultrasound transducer's position in space. Compounding the relative imprecision of conventional ultrasonographic systems is the fact that they generally determine position data relative to fixed objects adjacent to the patient, such as chest boards the patient is resting on, rather than relative to targets on the patient's body itself. The precision limitations of current ultrasonographic systems mean that practitioners must rely on external landmarks on the body to locate vertebra in need of treatment, which is prone to error.

Known ultrasonographic systems are typically not configured to automatically match new images of a patient's spine to previously acquired images of the patient's spine. The inability of conventional systems to effectively register new spinal images with previously acquired spinal images limits the practitioner's ability to accurately compare a given segment of the spine over time and to evaluate treatment effectiveness.

How acquired images are displayed by conventional ultrasonographic systems highlights another limitation of conventional systems. For example, conventional systems characteristically display acquired images on a two-dimensional screen. Even systems capable of representing acquired images in three-dimensional space generally do so on a two-dimensional screen, with the associated inherent limitations, and lack means to stereoscopically display the images in three-dimensional space.

Another drawback of conventional systems to examine and treat spinal conditions with ultrasound equipment relates to their inability to adequately extrapolate motion of the spine. Often, conventional ultrasonographic systems are limited to static images of the spine without an effective way to correlate different images of the spine when the patient moves to different positions. The inability to correlate images of the spine in different positions deprives the practitioner of important information regarding how the vertebrae move when flexing, extending, and/or rotating.

Thus, there exists a need for ultrasonographic systems that improve upon and advance the design of known ultrasonographic systems. Examples of new and useful ultrasonographic systems relevant to the needs existing in the field are discussed below.

Disclosure addressing one or more of the identified existing needs is provided in the detailed description below. Examples of references relevant to ultrasonographic systems include U.S. Patent Reference 20110021914. The complete disclosure of the referenced patent application is herein incorporated by reference for all purposes.

### SUMMARY

The present disclosure is directed to an ultrasonographic imaging system for examining and treating spinal conditions. In some examples, the system includes an ultrasound transducer probe configured to captured an ultrasound image, an ultrasound processor configured to receive data from the ultrasound transducer probe, a video capture card configured to receive the digital image from the ultrasound processor, an image processing system configured to process the digital image, the image processing system further including an image restoration module, an algorithm module, and a visualization module, a plurality of optical targets and a display device.

### BRIEF DESCRIPTION OF THE DRAWINGS

FIG. 1 is a schematic view of an ultrasonographic system for examining and treating spinal conditions consistent with the present invention.

FIG. 2 is a schematic view of a programmable computing device component of an image processing system of the ultrasonographic systems described herein.

FIG. 3 is a perspective view of an ultrasound transducer probe and optical targets.

FIG. 4 is a view of the optical targets placed on the spine of a patient.

FIG. 5 is a view of the optical targets placed on the spine of a patient.

### DETAILED DESCRIPTION

The disclosed ultrasonographic systems with become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, a variety of ultrasonographic system examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

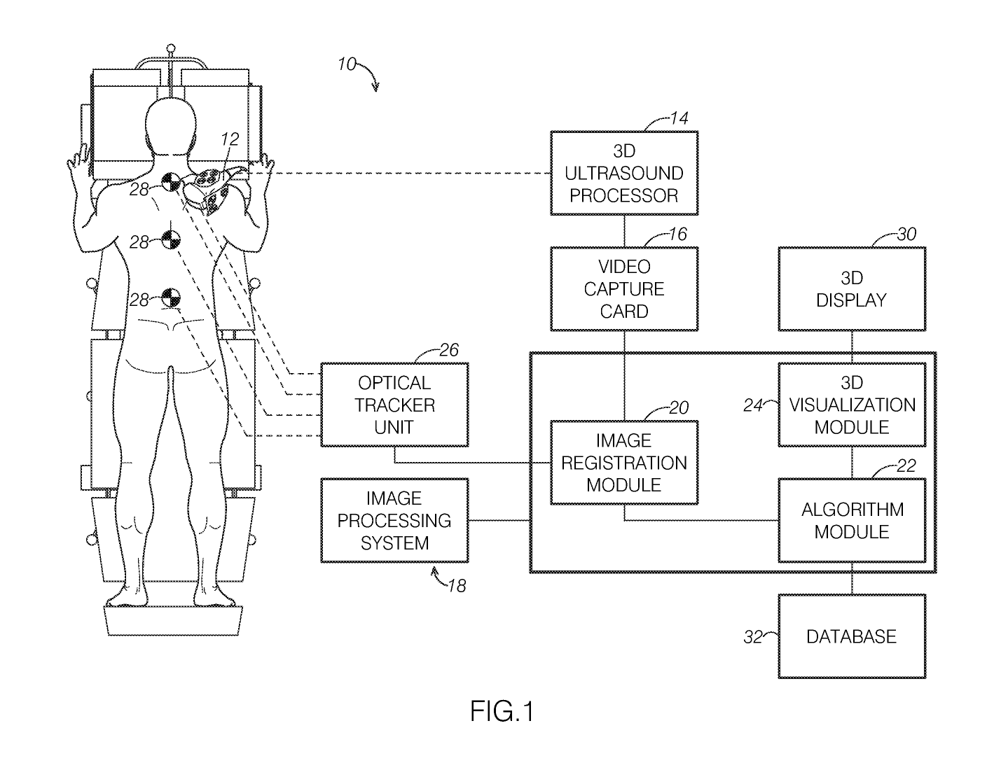

With reference to FIG. 1, an ultrasonographic system 10 for examining and treating spinal conditions will be described. The components of ultrasonographic system 10 will be listed and then explained in more detail below. As shown in FIG. 1, ultrasonographic system 10 includes an ultrasound transducer probe 12, a three-dimensional ultrasound processor 14, a video capture card 16, an image processing system 18, an optical tracker unit 26, optical targets 28, a stereoscopic three-dimensional display 30, and a database 32. Image processing system 18 includes an image registration module 20, an algorithm module 22, and a three-dimensional visualization module 24.

Before discussing the components of ultrasonographic system 10 in detail, some of the functions and capabilities of the system as a whole will be described to provide context to the system. As a variety of system examples are contemplated, the reader should understand that different system examples will provide different combinations of features and operating characteristics.

Ultrasonographic system 10 of FIG. 1 is configured to enable a practitioner to acquire images of a patient's spine in real-time with ultrasound transducer probe 12 and processor 14 without subjecting the patient to potentially harmful ionizing radiation. Further, ultrasonographic system 10 of FIG. 1 enables a practitioner to acquire images of the outer cortex of a patient's spine with high resolution on a real-time or substantially real-time basis.

Moreover, the ultrasonographic systems described herein are configured to automatically process acquired images of a user's spine into three dimensional images with the three-dimensional ultrasound system and imaging processing system 18. In some examples, the system is configured to stereoscopically display the images in three dimensions, such as with the 3D visualization module and the 3D stereoscopic display shown in FIG. 1.

As shown in FIG. 1, ultrasonographic system 10 utilizes optical tracking technology in the form of optical tracking unit 26 and optical targets 28 to precisely detect the patient's and ultrasound transducer probe's 12 position in space. However, additionally or alternatively to the optical tracking technology included in the example of FIG. 1, the ultrasonographic system may include magnetic positioning systems or attitude heading reference systems to detect the position of the patient, the transducer, or both. It is worth noting that that system 10 of FIG. 1 is configured to detect the position of the patient directly by the optical target positioned on the patient as opposed to merely detecting the position of a fixed object near the patient, such as a chest board or other stationary reference objects.

Additionally or alternatively, the ultrasonographic system may include an infrared scanning system configured to scan illuminated objects, such as patients, in three-dimensions. The infrared scanning system may include an infrared light projector, a camera or CMOS image sensor to detect the infrared light interacting with illuminated objects, and a microchip including computer executable instructions for spatially processing scanned objects. Suitable infrared scanning systems include the Light Coding™ system included in the Kinect™ gaming system. The infrared scanning system may supplement the optical tracking device and optical targets described above or may replace them in some applications.

Ultrasonographic system 10 of FIG. 1 affords the practitioner with highly precise information about the patent's position and the position of ultrasound transducer probe 12. Further, system 10 provides the practitioner with substantially real-time three dimensional images of a patient's spine by processing images generated by the 3D ultrasound system with image processing system 18 and displaying them on 3-D display 30. By combining precise position data and substantially real-time spinal image data, the inventive ultrasound systems described herein allow the practitioner to accurately locate internal features on a patient's spine, such as a particular vertebra in need of treatment. In fact, the systems described in this application enable a practitioner to locate internal features without having to rely on external landmarks on the body, which is a technique prone to error.

While certain examples of ultrasonic systems described herein display acquired images on a two-dimensional screen, system 10 of FIG. 1 stereoscopically displays acquired images in three-dimensional space. Stereoscopically displaying images of a patient's spine in three-dimensional space enables the practitioner to more accurately examine and treat the patient's spine.

Ultrasonographic system 10 of FIG. 1 is configured to automatically match new images of a patient's spine to previously acquired images of the patient's spine stored in database 32 with image registration module 20. The ability of system 10 in FIG. 1 to effectively register new spinal images with previously acquired spinal images allows a practitioner to accurately compare a given segment of the spine over time and to evaluate treatment effectiveness.

Ultrasonographic system 10 of FIG. 1 is configured to interpolate and/or extrapolate motion of the spine when the patient moves to different positions. System 10 interpolates and extrapolates spinal motion by registering different images of the spine acquired by ultrasound processor 14 to a common reference frame with image registration module 20. The ability of ultrasonographic system 10 to correlate images of the spine in different positions provides the practitioner with important information regarding how the patient's vertebrae move when flexing, extending, and/or rotating.

With reference to FIG. 1, the reader can see that ultrasonographic system 10 includes an ultrasound system having transducer probe 12 and a 3D ultrasound processor 14. Transducer probe 12 is in data communication with 3D ultrasound processor 14. In some examples, the 3D ultrasound processor is supported within the transducer probe and in other examples the components are separate from one another. Any conventional or later developed means of data communication between the transducer and the 3D ultrasound processor may be employed, such as wired communication and wireless communication.

Transducer probe 12 will in many examples be of a size suitable to be held in a practitioner's hand and moved over the patient's spine. Further, transducer probe 12 is configured to receive a plurality of optical targets 28 that aid optical tracker unit 26 in determining transducer probe's 12 positioning in space. The practitioner may move the transducer probe 12 over the entire posterior aspect of the spine or just an area of interest. In a known manner, transducer probe 12 interrogates the patient's spine with high frequency sound waves as schematically depicted in FIG. 1.

3D ultrasound processor 14 receives data from transducer probe 12 and generates three-dimensional images based on the data from transducer probe 12. In some examples, the system includes a 2D ultrasound processor instead of or in addition to the 3D ultrasound processor. The images generated by the 3D ultrasound processor 14 are sent to video capture card 16.

From video capture card 16, the three dimensional images generated by ultrasound processor 14 are sent to image processing system 18. In particular, as shown in FIG. 1, the images are sent to image registration module 20 of image processing system 18. Image registration module 20 is configured to correlate different images to a common reference frame. Any conventional or later developed image registration module may be used.

Working in conjunction with image registration module 20 is algorithm module 22 of image processing system 18. Algorithm module 22 includes computer executable instructions for coordinating the flow of data through image processing system 18 and to components in data communication with image processing system 18. For example, algorithm module 22 includes computer executable instructions for polling the database for previously acquired images and for sending registered image data to database 32 to be stored for later use. Additional instruction sets that may be programmed into the algorithm include instructions for delivering resolved image data to 3D visualization module 24 and/or for coordinating data inputs from optical tracker 26.

3D visualization module 24 may be any conventional or later developed software and/or hardware for generating three-dimensional visual data for display on a display device. In the example shown in FIG. 1, 3D visualization module 24 is configured to generate stereoscopic three-dimensional visual data for display on a device configured to stereoscopically display three-dimensional images. 3D display 30 may include any conventional or later developed accessories for stereoscopic viewing of the three-dimensional images, including 3D glasses and the like.

As shown in FIG. 1, optical tracker unit 26 is configured to optically track the position of one or more optical targets 28. Optical tracking devices 26 may be any known or later developed devices for detecting and correlating the position of optical targets 28, such as multiple two-dimensional imaging sensors or cameras.

In addition, optical tracker unit 26 is configured to determine the position of optical targets 28 as they move through space. In particular, optical tracker unit 26 is configured to determine the position of optical targets 28 about three axes and with six degrees of freedom. Optical tracker unit 26 may include multiple image sensors (not shown) and be configured to calculate the location of every optical target 28 through geometric triangulation. When more than two markers are grouped together to form a rigid-body target, it becomes possible to determine the target's orientation, yielding a total of six degrees of freedom.

Optical targets 28 may be conventional, specially developed, or later developed targets configured to cooperate with optical tracking unit 26. In some examples, the optical targets extend in three dimensions about three coordinate axes and include distinct targets representing each axis. In other examples, the optical target extends in three dimensions about six axes and includes distinct targets representing each of the six axes. The optical targets may be active, such as by emitting infrared signals to the optical target, or passive, such as including retro-reflective markers affixed to some interaction device.

Any number of optical targets 28 may be employed, with more optical targets 28 being used generally increasing the precision of the positional data. However, fully satisfactory results may be obtained with two optical targets 28. In some examples, a single optical target is used.

Additionally or alternatively, the ultrasonographic system may include precision-augmented tracking modules or optical tracker units when relaying positional data of the transducer probe and optical trackers back to the optical tracker unit. Such optical tracker units may employ solid-state gyroscopes to achieve this added measure of precision.

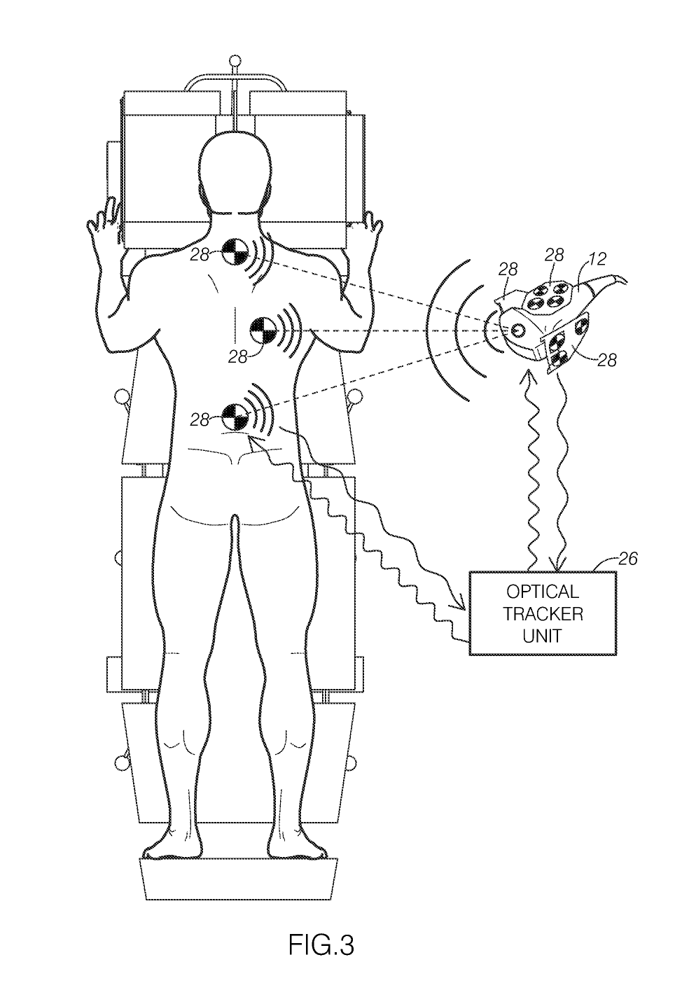

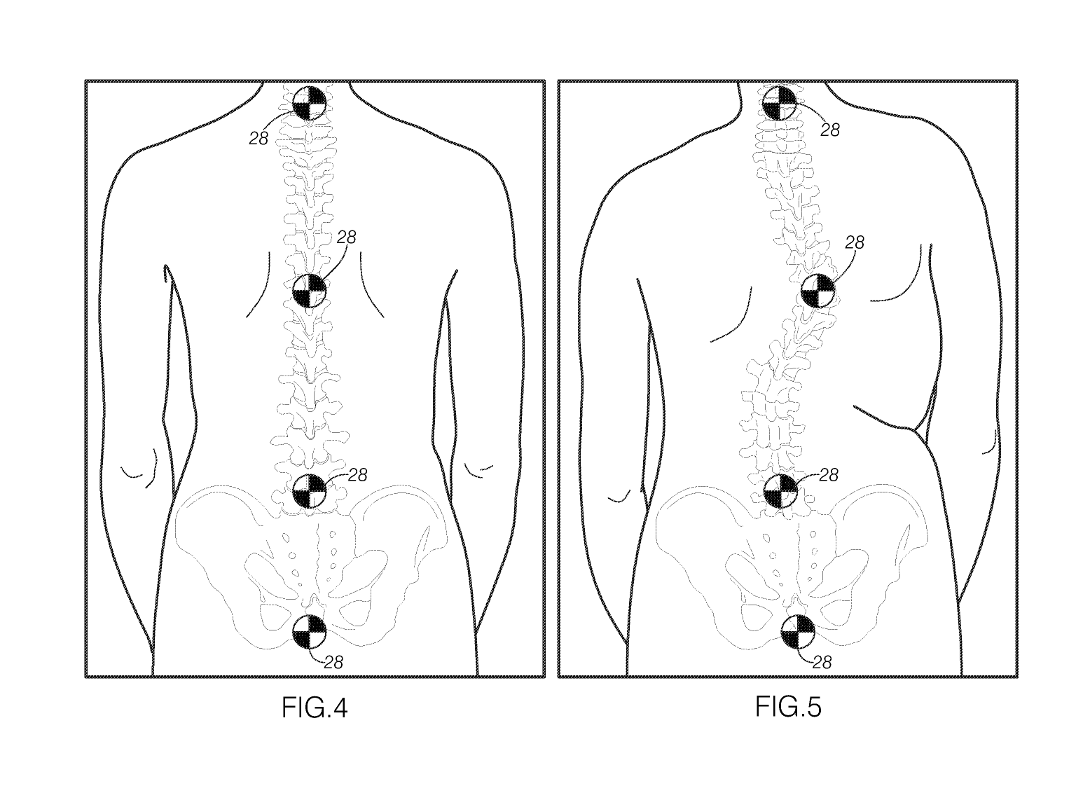

Turning attention to FIGS. 4 and 5, when ultrasonographic system 10 is utilized, the patient will have a plurality of optical targets 28 affixed to a desired location on the patient's back and there will be at least one optical target 28 attached to the transducer probe 12. The patient will be positioned on a specially configured mechanical table (see FIGS. 1 and 3), that allows the patient to be partially standing, yet still supported. The specially configured table enables the patient to be in a weight bearing, but still and stationary position during the imaging process. In some examples, the patient is instructed to move his torso to different positions to enable the practitioner to acquire images of the patient's spine in different positions and to interpolate and/or extrapolate motion of the spine.

As can be seen in FIG. 3, optical tracker unit 26 will detect the position of optical targets 28 affixed to the patient and the position of optical targets 28 affixed to transducer probe 12. The position data will be sent to image registration module 20 to correlate the position of the transducer probe 12 relative to the position of the patient in space. The position data will be further correlated to the images of the patient's vertebra in acquired images with image processing system 18.

One example of optical targets 28 being placed in various locations along the patient's spine is depicted in FIG. 4. The patient's spine is in a fixed position and the patient refrains from any movement or contortion of their body while resting in the specially configured mechanical table. An ultrasonographic scan of the patient's spine with ultrasound transducer probe 12 would register an image of the patient's spine as being substantially straight as shown in FIG. 4.

Referring now to FIG. 5, optical targets 28 can be seen in the same position along the patient's spine; however, the patient has now moved or contorted their spine from the substantially straight position shown in FIG. 4. Ultrasound transducer probe 12 is able to track this movement information in real-time for image processing by image processing system 18.

The real-time processing of positional information by system 10 allows for focused treatment based on 3D stereoscopic images of the patient's spine. Further, system 10 aids the practitioner in reviewing current and past images, which allows for frequent changes in the patient's treatment plan as opposed to waiting months for results obtained through other conventional methods.

In some examples, prior images of a patient's spine will be compared with more recent images of the spine to determine treatment effectiveness and how well the patient is healing over time. The algorithm module and the image registration module may cooperate to correlate the images acquired at different times with high precision. In particular, the position data of one acquired image may be precisely mapped with the position data of another image to enable the practitioner to readily correlate the anatomical position of each image and have confidence that each image corresponds to the same interior anatomical feature of the patient. In this manner, the practitioner need not rely on external landmarks on the patient's body to correlate different images over time, which can lead to errors.

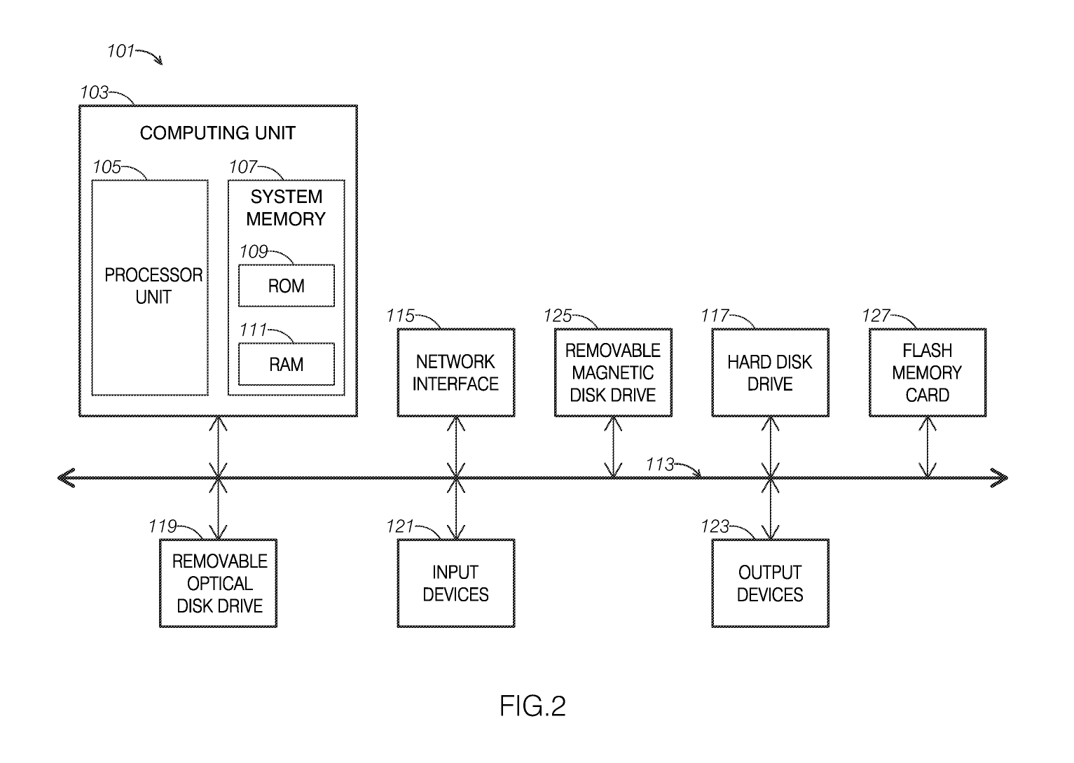

Turning attention to FIG. 2, a programmable computing device suitable for use as part of the image processing system will be described. While the following paragraphs describe one suitable example of an image processing system, the reader will understand that many different examples are contemplated. For example, image processing system 18 could include an embedded software system, a standalone personal computer, and/or a networked computer system.

Networked computer systems may suffer from bandwidth limitations with conventional network infrastructures, but future developments will likely alleviate those bandwidth issues. Standalone personal computers may not always providing the processing power to manage the data processing involved with the ultrasonographic systems described above. However, processing power improvements will certainly enable personal computers to handle the data processing involved in ultrasonographic systems described herein.

From the above discussion of ultrasonographic systems, those skilled in the art will recognize that various examples of the image processing system may be implemented using electronic circuitry configured to perform one or more functions. For example, with some embodiments of the invention, the image processing system may be implemented using one or more application-specific integrated circuits (ASICs). In some examples, however, components of various examples of the invention will be implemented using a programmable computing device executing firmware or software instructions, or by some combination of purpose-specific electronic circuitry and firmware or software instructions executing on a programmable computing device.

Accordingly, FIG. 2 shows one illustrative example of a computer 101 that can be used to implement various embodiments of the invention. As seen in this figure, computer 101 has a computing unit 103. Computing unit 103 typically includes a, processing unit 105 and a system memory 107. Processing unit 105 may be any type of processing device for executing software instructions, but will conventionally be a microprocessor device. System memory 107 may include both a read-only memory (ROM) 109 and a random access memory (RAM) 111. As will be appreciated by those of ordinary skill in the art, both read-only memory (ROM) 109 and random access memory (RAM) 111 may store software instructions to be executed by processing unit 105.

Processing unit 105 and system memory 107 are connected, either directly or indirectly, through a bus 113 or alternate communication structure to one or more peripheral devices. For example, processing unit 105 or system memory 107 may be directly or indirectly connected to additional memory storage, such as a hard disk drive 117, a removable optical disk drive 119, a removable magnetic disk drive 125, and a flash memory card 127. Processing unit 105 and system memory 107 also may be directly or indirectly connected to one or more input devices 121 and one or more output devices 123. Input devices 121 may include, for example, a keyboard, touch screen, a remote control pad, a pointing device (such as a mouse, touchpad, stylus, trackball, or joystick), a scanner, a camera or a microphone. Output devices 123 may include, for example, a monitor display, an integrated display, television, printer, stereo, or speakers.

Still further, computing unit 103 will be directly or indirectly connected to one or more network interfaces 115 for communicating with a network. This type of network interface 115, also sometimes referred to as a network adapter or network interface card (NIC), translates data and control signals from computing unit 103 into network messages according to one or more communication protocols, such as the Transmission Control Protocol (TCP), the Internet Protocol (IP), and the User Datagram Protocol (UDP). These protocols are well known in the art, and thus will not be discussed here in more detail. An interface 115 may employ any suitable connection agent for connecting to a network, including, for example, a wireless transceiver, a power line adapter, a modem, or an Ethernet connection.

It should be appreciated that, in addition to the input, output and storage peripheral devices specifically listed above, the computing device may be connected to a variety of other peripheral devices, including some that may perform input, output and storage functions, or some combination thereof. For example, the computer 101 will often be connected to the 3D ultrasound processor and transducer system. In addition to a 3D ultrasound unit, computer 101 may be connected to or otherwise include one or more other peripheral devices, such as a telephone.

The telephone may be, for example, a wireless “smart phone,” such as those featuring the Android or iOS operating systems. As known in the art, this type of telephone communicates through a wireless network using radio frequency transmissions. In addition to simple communication functionality, a “smart phone” may also provide a user with one or more data management functions, such as sending, receiving and viewing electronic messages (e.g., electronic mail messages, SMS text messages, etc.), recording or playing back sound files, recording or playing back image files (e.g., still picture or moving video image files), viewing and editing files with text (e.g., Microsoft Word or Excel files, or Adobe Acrobat files), etc. Because of the data management capability of this type of telephone, a user may connect the telephone with computer 101 so that their data maintained may be synchronized.

Of course, still other peripheral devices may be included with or otherwise connected to a computer 101 of the type illustrated in FIG. 2, as is well known in the art. In some cases, a peripheral device may be permanently or semi-permanently connected to computing unit 103. For example, with many computers, computing unit 103, hard disk drive 117, removable optical disk drive 119 and a display are semi-permanently encased in a single housing.

Still other peripheral devices may be removably connected to computer 101, however. Computer 101 may include, for example, one or more communication ports through which a peripheral device can be connected to computing unit 103 (either directly or indirectly through bus 113). These communication ports may thus include a parallel bus port or a serial bus port, such as a serial bus port using the Universal Serial Bus (USB) standard or the IEEE 1394 High Speed Serial Bus standard (e.g., a Firewire port). Alternately or additionally, computer 101 may include a wireless data “port,” such as a Bluetooth® interface, a Wi-Fi interface, an infrared data port, or the like.

It should be appreciated that a computing device employed according various examples of the invention may include more components than computer 101 illustrated in FIG. 2, fewer components than computer 101, or a different combination of components than computer 101. Some implementations of the invention, for example, may employ one or more computing devices that are intended to have a very specific functionality, such as a server computer. These computing devices may thus omit unnecessary peripherals, such as the network interface 115, removable optical disk drive 119, printers, scanners, external hard drives, etc. Some implementations of the invention may alternately or additionally employ computing devices that are intended to be capable of a wide variety of functions, such as a desktop or laptop personal computer. These computing devices may have any combination of peripheral devices or additional components as desired.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

I claim:

1. An ultrasonographic imaging system for examining and treating spinal conditions, comprising:
an ultrasound transducer probe configured to capture an ultrasound image;
an ultrasound processor configured to receive data from the ultrasound transducer probe and to generate a digital image from the captured ultrasound image;
a video capture card configured to receive the digital image from the ultrasound processor;
an image processing system configured to process the digital image, the image processing system further comprising:
an image restoration module configured to correlate different digital images to a common reference frame;
an algorithm module, the algorithm module having a set of computer executable instructions for coordinating the flow of data through the image processing system; and
a visualization module configured to generate display images for visual display;
a plurality of optical targets configured to provide positional data;
an optical tracker configured to determine the position of the plurality of optical targets; and
a display device configured to show display images.

2. The system of claim 1, wherein the ultrasound processor is configured to generate three-dimensional digital images based on data received from the ultrasound transducer.

3. The system of claim 1, wherein the ultrasound processor is configured to generate two-dimensional digital images based on data received from the ultrasound transducer.

4. The system of claim 1, wherein the visualization module includes software and hardware configured to receive three-dimensional data from the ultrasound processor and is configured to generate stereoscopic three-dimensional digital images.

5. The system of claim 1, wherein the algorithm module includes computer executable instructions for delivering resolved three-dimensional digital image data to the visualization module for coordinating data inputs from the optical tracker.

6. The system of claim 1, wherein the optical tracker is configured to optically track the positions of the plurality of optical targets as they move through space.

7. The system of claim 6, wherein the optical tracker is configured to determine the position of the plurality of optical targets about three coordinate axes and with six degrees of freedom.

8. The system of claim 1, wherein the plurality of optical targets are configured to reflect infrared signals for detection by the optical tracker.

9. The system of claim 1, wherein the plurality of optical targets are configured to attach to a patient's body.

10. The system of claim 1, wherein at least one of the plurality of optical targets is connected to the ultrasound transducer probe.

11. The system of claim 1, wherein the display device is configured to display stereoscopic three-dimensional digital images.

12. The system of claim 1, wherein a database is configured to store and recall previously captured digital images.

13. An ultrasonographic imaging system for examining and treating spinal conditions, comprising:
an ultrasound transducer probe configured to capture an ultrasound image;
an ultrasound processor configured to receive data from the ultrasound transducer and for generating a digital image from the captured ultrasound image;
a video capture card configured to receive the digital image from the ultrasound processor;
an image processing system configured to process the digital image;
a plurality of optical targets configured to provide positional data, the plurality of optical targets being configured to extend in three dimensions about three coordinate axes and includes distinct targets representing each coordinate axis and with six degrees of freedom, and where the optical targets reflect positional data to a plurality of optical trackers in one of the following forms of energy: light energy, sonic energy, and ultrasonic energy; and
a display device configured to show display images.

14. The system of claim 13, wherein the plurality of optical targets are configured to attach to a patient's body.

15. The system of claim 13, wherein the ultrasound processor is configured to generate three-dimensional digital images based on data received from the ultrasound transducer probe.

16. The system of claim 13, wherein at least one of the plurality of optical targets is connected to the ultrasound transducer probe.

17. The system of claim 13, wherein the display device is configured to display stereoscopic three-dimensional digital images.

18. The system of claim 13, wherein a database is configured to store and recall previously captured digital images.

19. The system of claim 13, wherein the optical targets are attached to a patient's body and to the ultrasound transducer probe.

20. The system of claim 13, wherein the optical tracker is configured to simultaneously track the position of the optical targets attached to a patient and the optical targets attached to the ultrasound transducer probe.
